(19) United States
(12) Patent Application Publication
(10) Pub. No.: US 2022/0304136 A1
(43) Pub. Date: Sep. 22, 2022

(54) USE OF MAGNETIC ELEMENTS TO SHAPE AND DEFOCUS CHARGED PARTICLE BEAMS

(71) Applicant: Varian Medical Systems, Inc., Palo Alto, CA (US)

(72) Inventor: Joshua McNEUR, Palo Alto, CA (US)

(21) Appl. No.: 17/204,657

(22) Filed: Mar. 17, 2021

(51) Int. Cl. H05H 7/04; A61N 5/10

(52) U.S. Cl. CPC H05H 7/04; A61N 5/1077; H05H 2007/045; H05H 2007/043; A61N 2005/1089; A61N 2005/1087

(57) ABSTRACT

A set of magnetic elements is used in the beamline of a charged particle-based radiation therapy machine instead of scattering foils. The set of magnetic elements is located between the exit of the linear accelerator and the isocenter or patient, and is used for shaping and defocusing a charged particle beam used for charged particle-based treatment modalities.

### BACKGROUND

The use of radiation therapy to treat cancer is well known. Radiation therapy involves using a radiation therapy machine to deliver a beam of high-energy particles into a target volume (e.g., a volume that includes a tumor or lesion) in a patient. In electron-based radiotherapy treatment modalities, for example, high-energy electrons are used.

Typically, electron-based treatment modalities are based on the generation and acceleration of an electron beam in a linear accelerator followed by spatial shaping of that electron beam via its interaction with thin foils known as scattering foils. The scattering foils are located between the exit of the linear accelerator and the patient. The foils are used to change the size and shape of the beam: as the electrons scatter through the foil material, they are broadened transversely relative to the beam axis. The foils thus serve the purpose of expanding the electron beam so that it has a uniform spatial profile by the time it reaches the isocenter.

However, as the electron beam traverses the scattering foils, it loses a significant fraction of its current due to scattering. As a result, the electron current that passes through the foils and reaches the patient is significantly less than the current that entered the foils. This loss of electrons reduces efficiency and limits the dose rate that can be delivered by the beam to the patient.

Also, because of the scattering process, the scattering foils will heat up due to the deposited energy from the traversing electron beam. The thermal strain on the scattering foils can damage or decrease the lifetime of the foils, and thus can increase operating and maintenance costs if the foils need to be repaired and replaced.

In Flash radiotherapy, a high radiation dose is delivered to the target volume within a short period of time. For example, in Flash radiotherapy, a beam may deliver at least 40 grays (Gy) in less than one second, and may deliver as much as 120 Gy per second or more. The increased electron current required to achieve such high dose rates means that the thermal strain on scattering foils is of particular concern for Flash radiotherapy: the deposited energy from the traversing electron beam can heat up the scattering foils to the extent that the foils might melt.

To summarize, scattering foils have disadvantages that include susceptibility to thermal strain and loss of efficiency, and these disadvantages in particular present challenges to realizing the necessary high electron currents required to achieve high dose rates for applications such as Flash radiotherapy.

### SUMMARY

Alternative beam shaping mechanisms that do not result in a reduction in efficiency and in the thermal issues and effects described above are desirable. There are clear benefits to replacing the scattering foils with another beamline element that serves the same function, for high dose rate applications in particular.

Embodiments according to the present invention provide these benefits.

Embodiments according to the present invention can be used with electron beams as well as other types of charged particle beams such as proton beams and ion beams.

In embodiments, a set of magnetic elements is used in the beamline of a radiation therapy machine instead of conventional scattering foils. The set of magnetic elements is located between the exit of the linear accelerator and the isocenter or patient, and is used for shaping and defocusing the beam used for treatment modalities based on charged particles. Because the magnetic elements do not intercept the beam, they result in no loss of beam current, and thus are better than scattering foils for realizing high dose rates. By eliminating the scattering foils, the magnetic elements also avoid the thermal problems caused by a high-current beam hitting a scattering foil. Furthermore, the set of magnetic elements provides an advantage not achievable with scattering foils: the field strength of the magnetic elements can be adjusted during treatment, which allows for live control of the beam size and shape, thereby introducing another degree of flexibility to radiation therapy planning and treatment.

Different kinds of magnetic elements are suitable as replacements for scattering foils in radiation therapy machines, including but not limited to solenoids, quadrupoles, and higher-order magnets (e.g., sextupoles and octupoles). In embodiments, the set of magnetic elements includes two quadrupole magnets (a quadrupole doublet) with tunable field strength. The use of two quadrupole magnets is an effective and efficient compromise between adding functionality and control of the beam shape and minimizing the added footprint due to the magnets. Including more than two quadrupoles in the set of magnetic elements can add functionality but has a larger footprint. Simulations demonstrate that the quadrupole doublet setup allows for defocusing of the beam at multiple energies and for considerable flexibility in beam shaping.

With very high energy electron beam radiotherapy (VHEE), preliminary research has indicated that focusing of the electron beam with a focal point near the isocenter can lead to reduced dose to healthy tissue while maintaining high dose to the target volume. A set of magnet elements as disclosed herein can be configured to realize this added benefit (e.g., by changing the magnetic field strengths in the quadrupole doublet).

These and other objects and advantages of embodiments according to the present invention will be recognized by one skilled in the art after having read the following detailed description, which are illustrated in the various drawing figures.

This summary is provided to introduce a selection of concepts that are further described below in the detailed description that follows. This summary is not intended to identify key features or essential features of the claimed subject matter, nor is it intended to be used to limit the scope of the claimed subject matter.

### BRIEF DESCRIPTION OF DRAWINGS

The accompanying drawings, which are incorporated in and form a part of this specification and in which like numerals depict like elements, illustrate embodiments of the present disclosure and, together with the detailed description, serve to explain the principles of the disclosure.

FIG. 1A is a block diagram showing selected elements of an example of a system for radiation therapy (a radiation therapy machine) in embodiments according to the present invention.

FIG. 1B is a block diagram showing selected elements of another example of a system for radiation therapy (a radiation therapy machine) in embodiments according to the present invention.

FIG. 2 is an example of a set of magnetic elements included in a system for radiation therapy (a radiation therapy machine) in embodiments according to the present invention.

FIG. 3 is a flowchart of an example of operations in a radiotherapy treatment method in embodiments according to the present invention.

### DETAILED DESCRIPTION

Reference will now be made in detail to the various embodiments of the present disclosure, examples of which are illustrated in the accompanying drawings. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

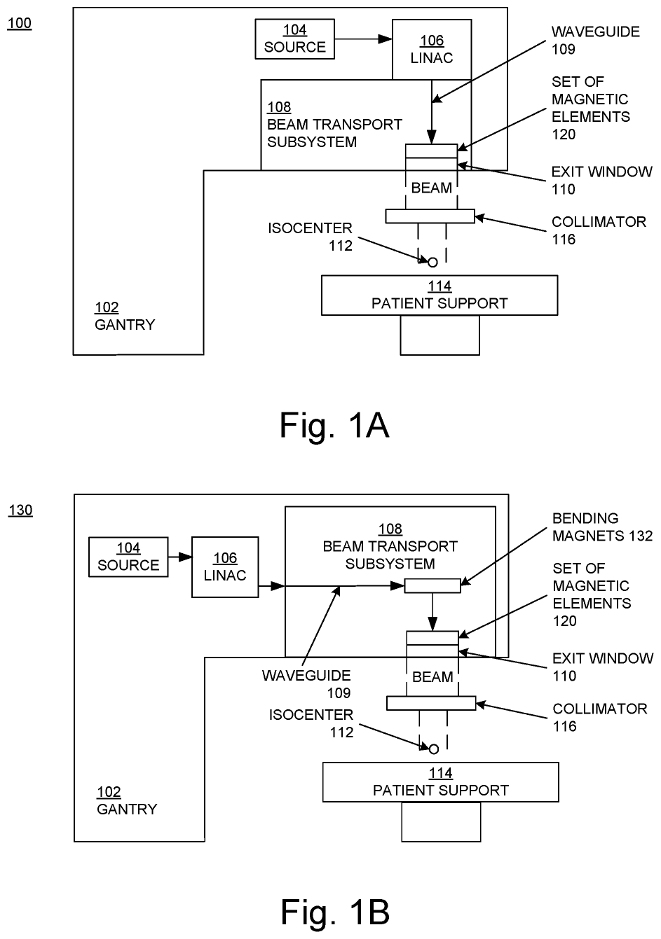

FIG. 1A is a block diagram showing selected elements of an example of a system 100 for radiation therapy (a radiation therapy machine) in embodiments according to the present invention. The system 100 can include elements in addition to or other than the elements shown in the figure and described or mentioned below.

In the example of FIG. 1A, the system 100 includes a movable gantry 102 that includes a particle source 104, a linear accelerator (LINAC) 106, and a beam transport subsystem 108. In an embodiment, the source 104 is a source of charged particles such as electrons, protons, or ions. A beam of charged particles from the source 104 is injected into the linear accelerator 106, which accelerates the charged particles (increases their energies), to an energy in the range of 1-2 MeV (million electron-volts) to up to 100 MeV or higher (even as high as 300 MeV). Charged particles having energies within that range are suitable for Flash radiotherapy. Embodiments according to the present invention are therefore suitable for the higher dose rates required for Flash radiotherapy as well as for the lower dose rates used for more conventional radiotherapy.

The charged particle beam subsequently enters the beam transport subsystem 108. In embodiments, the beam transport subsystem 108 includes a waveguide 109, which may be evacuated to form a vacuum. The beam transport subsystem 108 can include elements (not shown) such as beam steering and focusing coils that focus the charged particle beam to close to the central axis of the waveguide 109.

The beam transport subsystem 108 includes an exit window 110 that separates the internal environment (e.g., the vacuum) of the waveguide 109 from the atmosphere (air) outside the beam transport subsystem and waveguide.

The charged particle beam passes though the exit window 110 toward the isocenter 112 (toward a target volume in a patient on the patient support 114). A beam collimator 116 (e.g., a multileaf collimator) between the exit window 110 and the isocenter 112 can be used to shape the beam so that it more closely conforms to the shape of the target volume.

In embodiments according to the present invention, the beam transport subsystem 108 includes a set of magnetic elements 120 that replaces the scattering foils used in conventional radiation therapy machines. The magnetic elements 120 are used to shape and defocus the charged particle beam before the beam exits the beam transport subsystem 108 through the exit window 110. In an embodiment, the magnetic elements are adjacent to the exit window 110. In an embodiment, the magnetic elements 120 are the last component through which the charged particle beam passes before the beam reaches the exit window 110.

The set of magnetic elements 120 includes magnetic elements such as, but not limited to: solenoids, quadrupoles, and higher order magnets (e.g., sextupoles and octupoles). In embodiments, the set of magnetic elements 120 includes two quadrupole magnets (a quadrupole doublet) with tunable field strength.

The use of two quadrupole magnets is an effective and efficient compromise between adding functionality and control of the beam shape and minimizing the added footprint due to the magnets. Including one or more additional quadrupoles in the set of magnetic elements 120 can add functionality but results in a larger footprint. Simulations demonstrate that the quadrupole doublet setup allows for defocusing and shaping of the charged particle beam at multiple energies and for considerable flexibility in beam shaping.

When the charged particle beam enters the set of magnet elements 120, it is relatively narrow (e.g., a pencil beam). Defocusing and shaping of the charged particle beam using the set of magnetic elements 120 includes broadening the shape of the beam in transverse directions relative to the direction of the beam (the beam's longitudinal axis) and shaping the expanded beam.

The amount of broadening (dispersal) of the beam shape can be the same or different in the various transverse directions. That is, for example, the amount of broadening can be approximately the same in each of the transverse directions to create a beam having a roughly circular cross-section (when viewed down the beam axis). Alternatively, the amount of broadening can be different in the transverse directions to create a beam having an elliptical cross-section, for example. In the latter case, the desired shape can be created by using different magnetic field strengths. For example, in a quadrupole doublet, one of the quadrupoles can have one field strength, and the other can have a different field strength.

Increasing the number of quadrupoles to more than two can provide more flexibility in shaping the beam. Octupoles can be used to create a beam having a more complex cross-section, including non-Gaussian shapes such as squares or rectangles.

As mentioned above, the charged particles can have energies in the range of 1-2 MeV to up to 100 MeV or higher (even as high as 300 MeV), and charged particles having energies within that range are suitable for Flash radiotherapy. Beam rigidity increases as the energy of the charged particles increases. The magnetic field strengths of the set of magnetic elements 120 are high enough to handle beam rigidities for beam energies suitable for Flash radiotherapy. Embodiments according to the present invention are therefore suitable for the beam energies and dose rates required for Flash radiotherapy as well as for the beam energies and dose rates used in more conventional radiotherapy.

FIG. 1B is a block diagram showing selected elements of an example of a system 130 (a radiation therapy machine) for radiation therapy in other embodiments according to the present invention. The system 130 can include elements in addition to or other than the elements shown in the figure and described or mentioned below. Like numerals depict like elements in FIG. 1A.

Relative to the system 100 of FIG. 1A, the beam transport subsystem 108 of the system 130 includes bending magnets 132. The bending magnets 132 are used in, for example, radiation treatment machines that use very high energy beams and so require a longer linear accelerator that is mounted at a right-angle relative to the final direction of the beam. The bending magnets 132 direct (bend) the beam toward the set of magnetic elements 120, the exit window 110, and the isocenter 112. In the embodiments of FIG. 1B, the set of magnetic elements 120 are located as described above and so are downstream of the bending magnets 132, between the bending magnets and the exit window 110.

In both of the example systems of FIGS. 1A and 1B, the set of magnetic elements 120 circumvents the need for scattering foils and permits the utilization of high dose rate charged particle beams. For the purposes of charged particle beam shaping and defocusing, the set of magnet elements 120 has multiple advantages over scattering foils. Because the magnetic elements do not intercept the charged particle beam like a scattering foil, no current loss is incurred in the beam as it traverses the magnetic elements, and so there is no reduction in efficiency like that associated with scattering foils. Also, because the magnetic elements do not intercept the charged particle beam, the issues associated with heating of the scattering foil by the charged particle beam are avoided. Furthermore, unlike scattering foils, the magnetic elements allow the beam to be shaped in different ways so that the beam can be more closely matched to the shape of the target volume (e.g., tumor) prior to collimation.

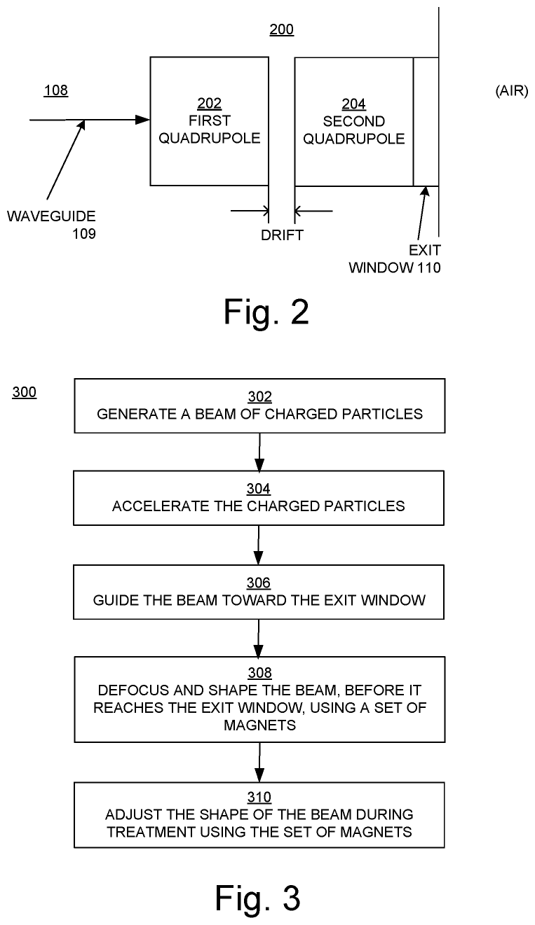

FIG. 2 is an example of a quadrupole doublet 200 in embodiments according to the present invention. The quadruple doublet 200 is an example of the set of magnetic elements 120 of FIGS. 1A and 1B. The quadrupole doublet 200 includes a first quadrupole 202 and a second quadrupole 204.

Simulations of the quadrupole doublet 200 demonstrate that such magnetic elements are suitable candidates for replacing scattering foils for beam energies for Flash radiotherapy as well as for more conventional beam energies, insofar as they can sufficiently disperse the beam without current loss and without the thermal problems associated with scattering foils. For example, a simulation was performed in which a field strength of six Tesla per meter (T/m) for the first quadrupole 202 and a field strength of 0.4 T/m for the second quadrupole 204 generated a beam with dimensions of 0.8 m by 1.1 m, measured one meter downstream of the second quadrupole 204. These field strengths were determined to be ideal for a six MeV beam, which is suitable for Flash radiotherapy. For this example, the simulation demonstrated that the quadrupole doublet 200 is able to defocus the beam to a spot size approaching one square meter about one meter after the exit window 110, which results in a sufficiently uniform transverse profile (cross-section) of the beam. The values just mentioned are examples only, and the invention is not limited to those values. Generally speaking, the disclosed invention is not limited to the use of quadrupole magnets or to the field strengths and beam energy used in the example simulation.

The quadrupoles 202 and 204 can have the same magnetic field strength, or they can have different magnetic field strengths. Significantly, the respective field strengths of the quadrupoles 202 and 204 are independently tunable. That is, the field strengths of the quadrupoles 202 and 204 can be changed or varied over time both independently of one another and remotely, with the quadrupoles in situ.

Using quadrupoles with tunable field strength provides the benefit of allowing for live, dynamic tuning and control of the shape and size of the charged particle beam during treatment, as opposed to having to stop treatment and then remove and replace scattering foils for this purpose. Furthermore, different configurations for different beam energies can be easily realized. Live control of the beam size and shape during treatment also introduces associated additional degrees of flexibility to radiation therapy planning. Those additional degrees of flexibility can be incorporated into radiation treatment plans, allowing the development of radiation treatment plans that result in more effective and efficient radiation treatments.

FIG. 3 is a flowchart 300 of an example of operations in a radiotherapy treatment method in embodiments according to the present invention. While the operations in FIG. 3 are presented as occurring in a certain order, the present invention is not so limited. The method of flowchart 300 can be implemented on or using the systems 100 and 130 of FIGS. 1A and 1B, respectively, and is described in conjunction with those figures.

In block 302, a beam of charged particles is generated by the source 104. In an embodiment, the charged particles are electrons; in another embodiment, the charge particles are protons; and in yet another embodiment, the charged particles are ions.

In block 304, the charged particles in the beam are accelerated by the linear accelerator 106.

In block 306, the beam is guided in the waveguide 109 in the beam transport subsystem 108 toward the exit window 110.

In block 308, before the beam reaches the exit window 110, the beam is defocused and shaped using the set magnetic elements 120.

In block 310, the shape and size of the beam are changed during treatment of a patient by varying the field strengths of the set of magnetic elements 120.

With very high energy electron beam radiotherapy (VHEE), preliminary research has indicated that focusing of the electron beam with a focal point near the isocenter can lead to reduced dose to healthy tissue while maintaining high dose to the target volume. A set of magnetic elements as disclosed herein can be configured to realize this added benefit (e.g., by changing the magnetic field strengths in the quadrupole doublet).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A radiotherapy treatment system, comprising:
a source of a beam of charged particles;
a linear accelerator coupled to the source and that accelerates the charged particles in the beam; and
a beam transport subsystem coupled to the linear accelerator and having an exit window, and comprising a plurality of magnetic elements located inside the beam transport subsystem between the linear accelerator and the exit window, wherein the plurality of magnetic elements is used to defocus the beam before the beam exits the beam transport subsystem.

2. The system of claim 1, wherein the charged particles comprise particles selected from the group consisting of: electrons; protons; and ions.

3. The system of claim 1, wherein the beam transport subsystem further comprises a plurality of bending magnets, and wherein the plurality of magnetic elements is between the plurality of bending magnets and the exit window.

4. The system of claim 1, wherein the plurality of magnetic elements is also used to shape the beam before the beam exits the beam transport subsystem.

5. The system of claim 1, wherein the plurality of magnetic elements comprises at least two quadrupole magnets.

6. The system of claim 5, wherein the quadrupole magnets have tunable field strengths.

7. The system of claim 1, wherein the plurality of magnetic elements comprises elements selected from the group consisting of: solenoids; sextupole magnets; and octupole magnets.

8. A radiotherapy treatment method, comprising:
generating a beam of charged particles;
accelerating the charged particles in the beam;
guiding the beam inside a waveguide toward an exit window that separates the inside of the waveguide from outside air; and
before the beam reaches the exit window, defocusing the beam using a plurality of magnetic elements.

9. The method of claim 8, wherein the charged particles comprise particles selected from the group consisting of: electrons; protons; and ions.

10. The method of claim 8, further comprising shaping the beam using the plurality of magnetic elements.

11. The method of claim 8, wherein said guiding comprises bending the beam using a plurality of bending magnets, wherein the plurality of magnetic elements is between the plurality of bending magnets and the exit window.

12. The method of claim 8, wherein the plurality of magnetic elements comprises at least two quadrupole magnets.

13. The method of claim 12, further comprising changing a cross-sectional shape and size of the beam during treatment of a patient by varying field strengths of the quadrupole magnets.

14. The method of claim 8, wherein the plurality of magnetic elements comprises elements selected from the group consisting of: solenoids; sextupole magnets; and octupole magnets.

15. A radiotherapy treatment method, comprising:
generating a beam of charged particles;
accelerating the charged particles in the beam;
guiding the beam inside a waveguide toward an exit window that separates the inside of the waveguide from outside air; and
before the beam reaches the exit window, using a plurality of magnetic elements to change a cross-sectional shape and size of the beam during treatment of a patient by varying field strengths of the plurality of magnetic elements.

16. The method of claim 15, wherein the charged particles comprise particles selected from the group consisting of: electrons; protons; and ions.

17. The method of claim 15, further comprising defocusing the beam using the plurality of magnetic elements.

18. The method of claim 15, wherein said guiding comprises bending the beam using a plurality of bending magnets, wherein the plurality of magnetic elements is between the plurality of bending magnets and the exit window.

19. The method of claim 15, wherein the plurality of magnetic elements comprises at least two quadrupole magnets.

20. The method of claim 15, wherein the plurality of magnetic elements comprises elements selected from the group consisting of: solenoids; sextupole magnets; and octupole magnets.
